(19) United States
(12) Patent Application Publication
(10) Pub. No.: US 2010/0132704 A1
(43) Pub. Date: Jun. 3, 2010

(54) BLISTER PACK AND APPLICATOR THEREFOR

(75) Inventors: Zoran Djinovic, Wiener Neustadt (AT); Herbert Weissenböck, Feistritz am Wechsel (AT); Martin Milojkovic, Wien (AT)

Correspondence Address: CHAPMAN AND CUTLER, 111 WEST MONROE STREET, CHICAGO, IL, 60603, US

(73) Assignee: CROMA-PHARMA GMBH, Korneuburg (AT)

(21) Appl. No.: 12/451,723

(22) PCT Filed: Jun. 2, 2008
(86) PCT No.: PCT/AT2008/000190

§ 371 Date: Nov. 25, 2009

(30) Foreign Application Priority Data
Jun. 1, 2007 (AT) A 876/2007

(51) Int. Cl. A61M 15/00; B65D 73/00; A61M 35/00

(52) U.S. Cl. 128/203.15; 206/462; 604/296

(57) ABSTRACT

In a container (3) for a plurality of single doses of a fluid active substance, in which the single doses are separately packaged in a gas-tight manner and can be released separately, the container (3) comprises a substantially flat carrier film, which carries a plurality of gas-tight cavities (33) including discharge channels (32) oriented to the periphery of the carrier film and opening at a distance from the periphery of the carrier film. Incisions (30) departing from the periphery and ending transversely to the discharge channels (32) at a distance from the same are provided, which discharge channels (32) are releasable by tearing and separating transversely to the axis of the discharge channels (32) upon bending and pulling on the tabs formed by the incisions (30).

The invention relates to a container for a plurality of single doses of a fluid active substance, in which the single doses are separately packaged in a gas-tight manner and can be released separately, as well as an applicator for such containers.

Active substances and, in particular, pharmaceutical substances can be conditioned in various administration forms. In the event of tablets, blister packages are, for instance, known, wherein the respective tablet can be pressed out of the package by applying a pressing power. For providing fluids, it has already been proposed, for instance in WO 03/073907 A2, to form a plurality of cavities in a disc-shaped carrier, with the fluid stored in such a cavity being released by the application of a pressing power while tearing the respective wall of the cavity. As in correspondence with that proposal, such devices are, therefore, above all suitable for providing detergents in dishwashers or the like, where precise dosing is not really the point and the random splashing of fluid is, moreover, irrelevant in view of the subsequent rinsing with rinse water.

For providing pharmaceutical substances, US 2006/0163269 A1, in particular, has already proposed to form a carrier strip with a plurality of cavities each covered by an adhesive tape. To release the active substances contained in the cavities and usually present in solid form, a portion of the cover film is each pulled off so as to render the uncovered substance accessible. Configurations of this type are relatively unsuitable for fluids and, in particular, are not suitable to ensure the formation of drops as would, for instance, be required for providing eye drops. The use of such a device for fluids would, moreover, involve the risk of contaminating the outlet openings, since a selective release is not readily feasible. In WO 96/06581, the release of active substances by the destruction of a cover film is achieved in that an appropriate pressing power is exerted on the bottom of the cavity so as to destroy the cover film, which may occasionally cause tearing of the cover film, thus contaminating the active substance with particles of the cover film. That device too is unsuitable for the selective and dosed dispensing of drops and, therefore, cannot be universally used. U.S. Pat. No. 6,443,307 B1, in which the cover film of a cavity is opened by a punch knife, also is only suitable to a limited extent for the selective dispensing of fluids, since impurities may form at the punch edges, particularly with accordingly sensitive substances, thus seriously jeopardizing the effectiveness of the active substance and the safety of its application, for instance when using eye drops.

The invention now aims to provide a container of the initially defined kind, by which a plurality of single doses can be provided in a manner that drops can each be formed individually, that contaminations during the opening of the respective single packages will, furthermore, be safely avoided, and that finally even sensitive fluids and, in particular, oxidation-sensitive fluids can be stored over extended periods of time, wherein accordingly tight materials are required for the limitation of the cavities receiving the single doses. Active substances which are, for instance, especially sensitive to oxidation, among others, include proteins, peptides, vitamins or prostaglandins, wherein, in particular, vitamin A, glucocorticoids, vegetable oils, carotinoids and lipids as well as compounds carrying thiol groups will require particularly resistant and gas-tight packages in order to prevent oxidation. Bearing in mind all of these prerequisites, the container according to the invention is to guarantee a suitable long-time stability of the single doses and to be usable without contaminating corresponding applicators.

To solve this object, the container according to the invention essentially consists in that the container comprises a substantially flat carrier film, which comprises a plurality of gas-tight cavities including discharge channels oriented to the periphery of the carrier film and opening at a distance from the periphery of the carrier film, that incisions departing from the periphery and ending transversely to the discharge channels at a distance from the same are provided, said discharge channels being releasable by tearing and separating transversely to the axis of the channels upon bending and pulling on the tabs formed by the incisions. Tearing will ensure opening without particle-contamination, and the gas-tight configuration will, in particular, safeguard that no oxygen will enter the cavities. The substantially flat carrier film may be configured in the form of a disc designed in any manner whatsoever and having a polygonal or even circular periphery, as in correspondence with a preferred configuration. Due to the fact that discharge channels oriented from such gas-tight cavities to the periphery of the carrier film and opening at a distance from the periphery of the carrier film are provided, it has become possible to adapt the channel configurations to the respective fluid to be dispensed in such a manner as to cause the formation of drops with the correct positioning of the container on the opened channel, thus enabling the selective and careful release of the fluid. Fluid in this context comprises both liquid and gaseous substances to be made available in an accordingly dosed manner by the suitable dimensioning of the channel. The provision of incisions about the periphery of the carrier film transversely to the discharge channels and ending at a distance of the latter enables said discharge channels to be opened by tearing, whereby the respective tearing edge is effected transversely to the respective channel axis and the accordingly deformed tab, which is delimited by such an incision, will subsequently remain connected with the carrier film such that no possibly contaminated part need to be disposed of. The disposal may rather be performed for the plurality of single doses together with the container. As compared to the known methods in which opening is to be effected by punching and pulling off adhesive films, the tearing open of the channel offers the advantage that the tearing edges are free of residues and, therefore, no contamination of the fluid discharged through the discharge channels need to be feared. After all, no adhesion surfaces are exposed or chips or the like depending on the cutting quality of punching knives are formed.

In a particularly advantageous manner, the configuration may be devised such that the gas-tight cavities and the discharge channels are delimited by a multilayer film structure so as to ensure the required gas-tightness. The selection of appropriate materials in this context will, at the same time, ensure that any evaporation of fluids from the containers will be safely avoided even under negative pressure as prevails, for instance, in accordingly decompressed aircraft cabins. The stable configuration of the carrier and the film delimiting the gas-tight cavities in this respect may also be devised such that the whole blister package can be sterilized by the application of vapour, or can be exposed to irradiation, without involving the destruction of the package. Finally, the surface in the immediate vicinity of the openings of the channels may be coated so as to guarantee absolute sterility, whereby the stable configuration is also apt for use in mechanically operated applicators.

The configuration is advantageously devised such that the substantially flat carrier film has a circular contour and the discharge channels are arranged in a substantially radial manner, wherein the discharge channels in a particularly advantageous manner have clear cross sections that are suitable for the formation of liquid drops.

In order to ensure the safe tearing open of the discharge channels, the configuration is advantageously devised such that the incisions, departing from the periphery of the carrier, are configured to extend substantially radially inwards transversely to the periphery over a first section and substantially in the peripheral direction and transversely to the discharge channels in a further section, said incisions ending in the vicinity of the discharge channels. When using mechanical applicators, a high storage stability and a high operational safety will be achieved in that the carrier film and/or the films delimiting the cavities and discharge channels, respectively, are designed as elastically prestressed films or elastically prestressed compound films.

The applicator according to the invention for such containers is essentially characterized in that a displacement drive or a rotation drive is provided for the carrier film, by the aid of which a discharge channel can be moved into a position in alignment with said discharge channel of an outlet opening of the applicator, and that means for gripping a tab adjacent the respective discharge channel and delimited by incisions are provided, which open the adjacent discharge channel in the position in alignment with the outlet opening of the applicator, while forming a tearing edge extending transversely to the discharge channel. Such an applicator is basically suitable for polygonal carrier films, yet preferably for carrier films having substantially circular contours, wherein the displacement or rotation drive each moves a discharge channel to an outlet opening in a manner that the discharge channel and the outlet opening will be aligned with each other. At the same time, means for gripping the tearing tabs are arranged within the applicator, which, during such rotational movement, will cause the discharge channel to open by tearing the tab, so that a discharge channel tearing edge extending transversely to the channel will reach a position in alignment with the outlet opening. If any contamination of the outlet opening by the fluid substances is to be likewise avoided during such application, the use of the applicator is to be only enabled in special positions in which the fluid or fluid drops are able to emerge without contacting the outlet opening. This will, in particular, be ensured in that a position sensor is provided, which enables the tearing open of the discharge channels only in a defined position relative to an application site and, in particular, in a substantially vertical position and/or a position recognizing the application site, as in correspondence with a preferred further development.

In order to be able to discharge the contents of the cavities completely, the configuration is preferably devised such that cheek plates acting transversely to the carrier cooperate with cavities whose discharge channels are moved into a position in alignment with the outlet opening.

A high degree of operational safety and a number of further simplifications in handling will be achieved in that the applicator comprises a freely programmable switching device and at least one display, by the aid of which time-specific or operation-specific data such as, e.g., the expiration of the effectiveness of the active substances, a low residual energy of the energy storage, minimum and/or maximum temperatures detected by temperature sensors, the time of day, or reminder signals can be displayed.

In the following, the invention will be explained in more detail by way of exemplary embodiments schematically illustrated in the drawing. Therein,

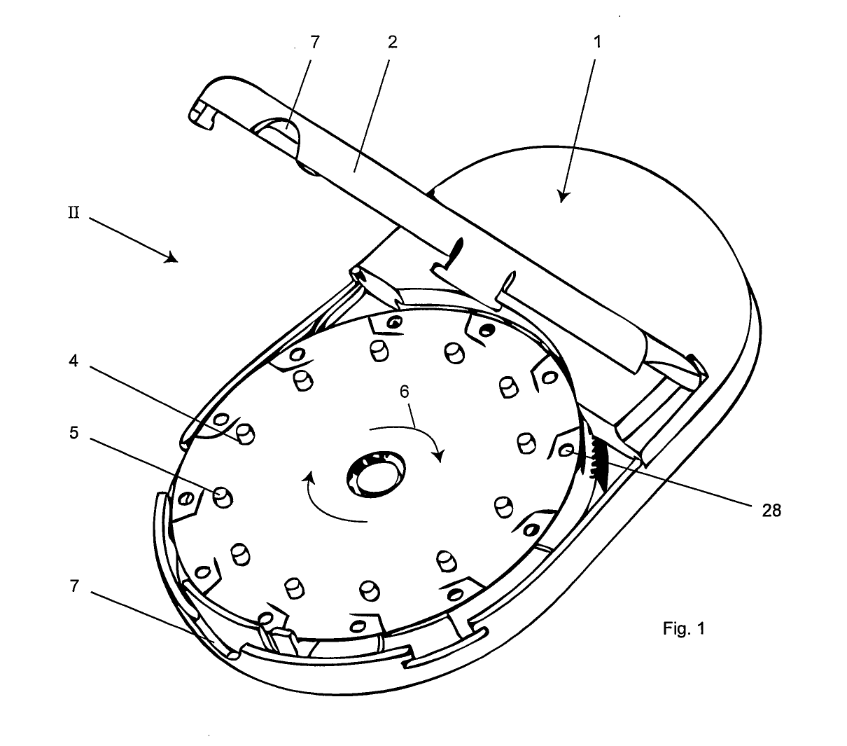

FIG. 1 illustrates a configuration of the applicator with the lid opened and a container inserted;

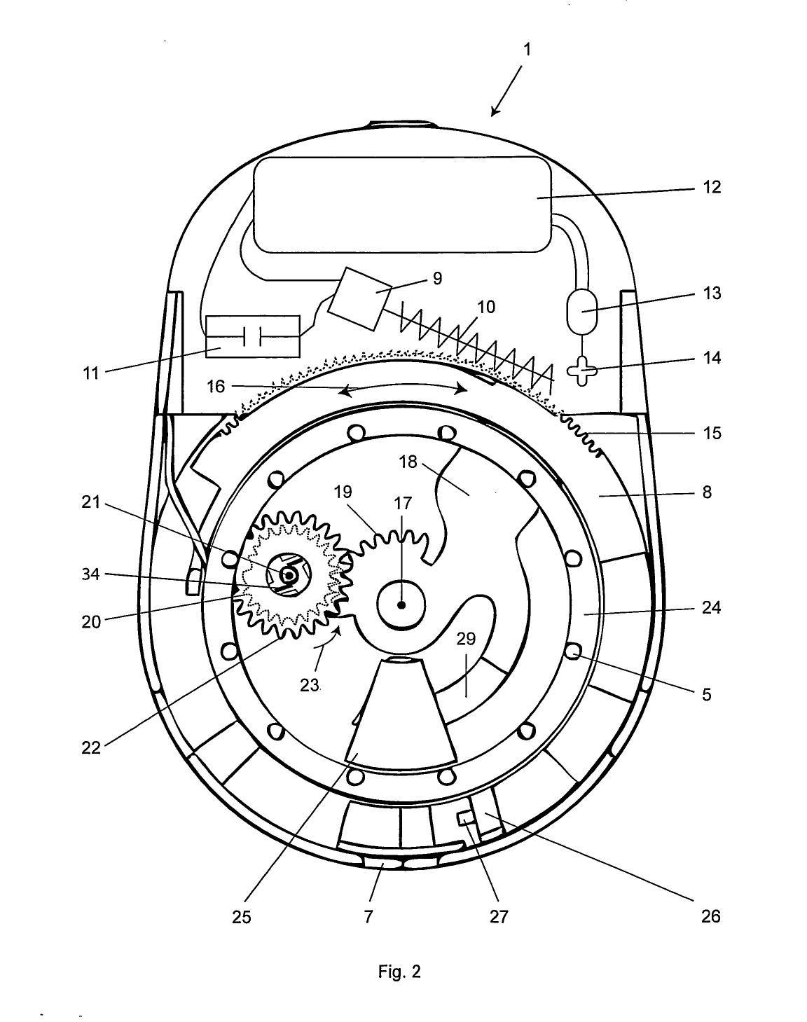

FIG. 2 is a view in the sense of arrow II of FIG. 1, with the container removed and the lid taken off;

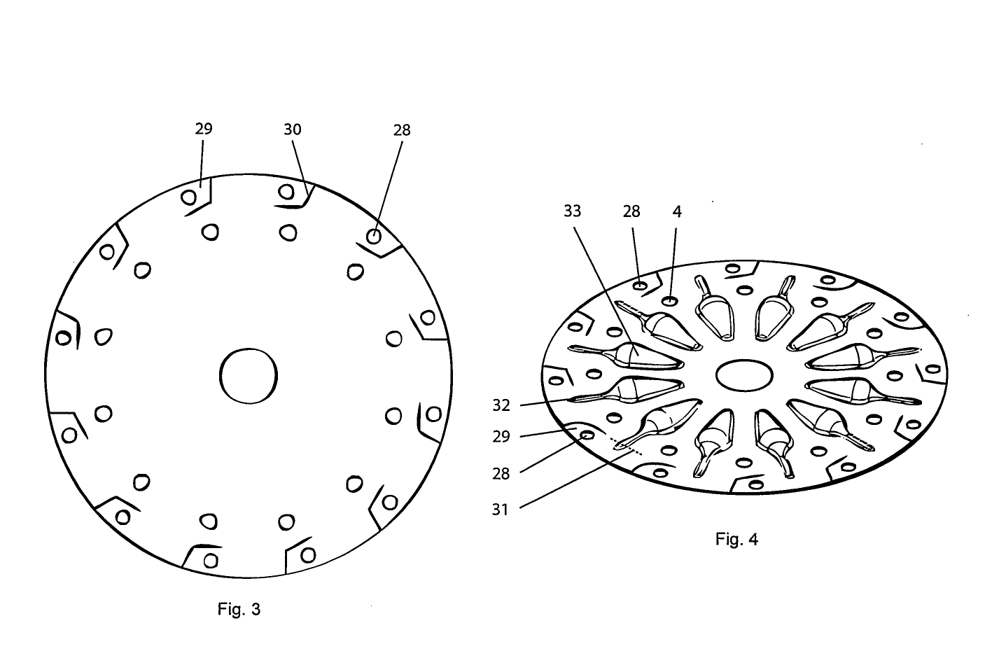

FIG. 3 is a top view on the container; and

FIG. 4 is a bottom view of the container including gas-tight cavities.

In FIG. 1, the housing of an applicator is denoted by 1, comprising a foldable lid 2. A container or carrier is denoted by 3, said container being visible in the top view and the cavities, which are not illustrated in FIG. 1, being merely visible in the bottom view in FIG. 4. The container includes a plurality of recesses or cavities 4 which cooperate with tenons 5 for transporting the container in the sense of arrow 6. An opening of the housing 1, via which active substances can be discharged, is denoted by 7.

From the illustration according to FIG. 2, in which the entire lid 2 has been removed together with the rear part of the housing, it is apparent that a motor 9 including a worm 10 is provided for driving a first annular disc 8. The energy supply is schematically indicated by a battery 11, wherein a display is denoted by 12, which is controlled by a freely programmable switching mechanism 13. By 14, a sensor is denoted by which the position of the housing 1 can be detected and evaluated in the freely programmable switching mechanism 13.

The ring 8, which carries a sprocket wheel 15, is moved by the worm 10 in the sense of double arrow 16, whereby, about the central axis 17, via a radially inwardly extending arm 18, which is connected with the ring 8, and a sprocket wheel 19, a toothed wheel 20 indicated in broken lines is driven to rotate about an axis 21. This toothed wheel 20 is connected with the toothed wheel 22 via a ratchet 34 such that the toothed wheel 22, due to the ratchet 34, is merely driven in the sense of arrow 23, yet not in the counter-direction. In this manner, the toothed wheel 22 will each stepwisely drive the ring 24, which is equipped with an internal toothing and carries the tenons 5 for the containers, thus each moving a new cavity to the cheek plate 25. The ring 24 is connected with an arm 26 which, by a tenon 27, will each grip the recesses of the container, which are denoted by 28 in FIG. 1, to thereby initiate the tearing movement. When pivoting the arm 18, a ramp 29 is guided to below the cheek plate 25 in such a manner as to cause the cheek plate 25 to be lifted as illustrated in FIG. 2 and exert an appropriate pressing power on the cavity of the container, as is more clearly illustrated in FIGS. 3 and 4.

In FIGS. 3 and 4, recesses 28 are provided in tabs 29, which can be grasped by the arm of the applicator as is apparent from FIG. 2, to be thereby bent up along incisions 30 in order to subsequently each expose discharge channels 32 by tearing open, as indicated in FIG. 4 by broken line 31. These discharge channels 32, by a rotational movement of the container, will each reach a position in alignment with the outlet opening 7, as is apparent from FIG. 1, the associated cavities being each denoted by 33. The containers themselves may be substantially disc-shaped and have multilayer structures, wherein the content of each container can be pressed out by the cheek plate 25 in the position of the discharge channels 32 each aligned with the outlet opening 7.

In order to ensure that the active substance will not contaminate the outlet opening 7 during such pressing out, the respective position of the applicator is determined by the sensor 14 and the release of the driving connection is electronically effected only under accordingly defined conditions.

In addition to current operating conditions, also the remaining number of not yet pressed-out cavities can, of course, be indicated on the display 12, wherein a number of additional operation-specific data such as, for instance, the expiration of the effectiveness of the active substances or the residual energy of the energy storage 11 may likewise be indicated in a simple manner.

Additional sensors will also enable the detection of the temperature and, if desired, the storage of minimum and/or maximum values.

A substantial advantage of the device according to the invention consists in that the fluid can also be discharged without pressure if appropriate drops are formed.

The container is gas-tight and may be designed to be UV-resistant.

1. A container for holding a plurality of single doses of a fluid active substance, in which the single doses are separately packaged in a gas-tight manner and are separately releasable, the container comprising:
a substantially flat carrier film carrying a plurality of gas-tight cavities, each of said cavities comprising a discharge channel oriented to a periphery of the carrier film and opening at a distance from the periphery of the carrier film, and
a plurality of incisions extending from the periphery of the carrier film, each of said incisions ending transversely to a respective one of the discharge channels at a distance from the respective discharge channel, each of said incisions delimiting a respective tab formed at the periphery of the carrier film, wherein
each of the discharge channels is releasable by tearing and separating transversely to an axis of the discharge channel upon bending and pulling on the respective tab formed by the incision.

2. A container according to claim 1, wherein the gas-tight cavities and the discharge channels are delimited by a multilayer film structure.

3. A container according to claim 1, wherein the substantially flat carrier film has a circular contour and the discharge channels are arranged in substantially radial positions around the circular contour.

4. A container according to claim 1, wherein the discharge channels have clear cross sections shaped for formation of liquid drops.

5. A container according to claim 1, wherein the incisions extending from the periphery of the carrier film are configured to extend substantially radially inwards transversely to the periphery of the carrier film over a first section, and substantially in a peripheral direction and transversely to the discharge channels in a further section, said incisions ending in a vicinity of the discharge channels.

6. A container according to claim 1, wherein the carrier film is an elastically prestressed film or an elastically prestressed compound film.

7. An applicator for a container for holding a plurality of single doses of a fluid active substance, in which the single doses are separately packaged in a gas-tight manner and are separately releasable, the container having a substantially flat carrier film carrying a plurality of gas-tight cavities, each of said cavities comprising a discharge channel oriented to a periphery of the carrier film and opening at a distance from the periphery of the carrier film, and a plurality of incisions extending from the periphery of the carrier film, each of said incisions ending transversely to a respective one of the discharge channels at a distance from the respective discharge channel, each of said incisions delimiting a respective tab formed at the periphery of the carrier film, wherein each of the discharge channels is releasable by tearing and separating transversely to an axis of the discharge channel upon bending and pulling on the respective tab formed by the incision,
said applicator comprising:
a displacement drive or a rotation drive for driving movement of the carrier film,
an outlet opening of the applicator, and
means for gripping the respective tab adjacent the respective discharge channel, wherein
the displacement drive or rotation drive is adapted to move the respective discharge channel into a position in alignment with the outlet opening, and
said means for gripping opens the respective discharge channel in the position in alignment with the outlet opening of the applicator, forming a tearing edge extending transversely to the respective discharge channel.

8. An applicator according to claim 7, further comprising cheek plates acting transversely to the container and cooperating with cavities whose discharge channels are moved into a position in alignment with the outlet opening.

9. An applicator according to claim 7, further comprising a position sensor adapted to enable tearing open of the discharge channels only in a defined position relative to an application site, said position being one or more of a vertical position and a position recognizing the application site.

10. An applicator according to claim 7, further comprising a freely programmable switching device and at least one display for displaying time-specific or operation-specific data.

11. An applicator according to claim 10, wherein the data is one or more of expiration of effectiveness of the active substances, low residual energy in energy storage, minimum temperatures detected by temperature sensors, maximum temperatures detected by temperature sensors, time of day, and reminder signals.

12. A container according to claim 2, wherein the multilayer film structure is an elastically prestressed film or an elastically prestressed compound film.

13. A container according to claim 2, wherein the substantially flat carrier film has a circular contour and the discharge channels are arranged in substantially radial positions around the circular contour.

14. A container according to claim 2, wherein the discharge channels have clear cross sections shaped for formation of liquid drops.

15. A container according to claim 3, wherein the discharge channels have clear cross sections shaped for formation of liquid drops.

16. A container according to claim 2, wherein the incisions extending from the periphery of the carrier film are configured to extend substantially radially inwards transversely to the periphery of the carrier film over a first section, and substantially in a peripheral direction and transversely to the discharge channels in a further section, said incisions ending in a vicinity of the discharge channels.

17. A container according to claim 3, wherein the incisions extending from the periphery of the carrier film are configured to extend substantially radially inwards transversely to the periphery of the carrier film over a first section, and substantially in a peripheral direction and transversely to the discharge channels in a further section, said incisions ending in a vicinity of the discharge channels.

18. A container according to claim 4, wherein the incisions extending from the periphery of the carrier film are configured to extend substantially radially inwards transversely to the periphery of the carrier film over a first section, and substantially in a peripheral direction and transversely to the discharge channels in a further section, said incisions ending in a vicinity of the discharge channels.

19. A container according to claim 2, wherein the carrier film is an elastically prestressed film or an elastically prestressed compound film.

20. A container according to claim 3, wherein the carrier film is an elastically prestressed film or an elastically prestressed compound film.
